(19) United States
(12) Patent Application Publication
(10) Pub. No.: US 2019/0160117 A1
(43) Pub. Date: May 30, 2019

(54) METHOD AND TOPICAL COMPOSITION FOR MODIFICATION OF A SKIN MICROBIOME

(71) Applicant: Access Business Group International LLC, Ada, MI (US)

(72) Inventors: Greg HILLEBRAND, Whitehall, MI (US); Brandon IKER, Grand Rapids, MI (US); Pedro DIMITRIU, Vancouver (CA); William MOHN, Vancouver (CA)

(21) Appl. No.: 16/203,731

(22) Filed: Nov. 29, 2018

Related U.S. Application Data
(60) Provisional application No. 62/592,158, filed on Nov. 29, 2017.

(51) Int. Cl. A61K 35/74; A61K 9/00; A61Q 19/02; A61Q 19/08; A61K 8/99; A61P 17/00

(52) U.S. Cl. CPC A61K 35/74; A61K 9/0014; A61P 17/00; A61Q 19/08; A61K 8/99; A61Q 19/02

(57) ABSTRACT

A method is disclosed. The method is generally useful for modifying a skin microbiome. The method comprises administering a topical composition to a subject's skin. A topical composition is also disclosed. The topical composition may be used for the method. The topical composition comprises a population of microorganisms, a component obtained from the population of microorganisms, or a combination thereof. The population of microorganisms is generally a Corynebacterium species. The Corynebacterium species comprises at least about 90%, optionally at least about 97%, sequence identity to a 16S rRNA sequence (SEQ ID NO: 1).

### CROSS-REFERENCE TO RELATED APPLICATIONS

This application claims priority to and all advantages of U.S. Provisional Patent Appl. No. 62/592,158 filed on 29 Nov. 2017, the contents of which is hereby incorporated by reference.

### SEQUENCE LISTING

The subject application contains a Sequence Listing which has been submitted electronically in ASCII format and is hereby incorporated by reference in its entirety. Said ASCII copy, created on 27 Nov. 2017, is named WN3376_ST25.txt and is 4,096 bytes in size.

### FIELD OF THE INVENTION

The present invention generally relates to a method for modifying a subject's skin microbiome using a topical composition. The present invention also relates to a topical composition. The topical composition can be used with the method. Each of the method and the topical composition is associated with a Corynebacterium species having a particular 16S rRNA sequence (SEQ ID NO: 1) as described herein.

### DESCRIPTION OF THE RELATED ART

Skin microbiome (or flora) typically refers to the microorganisms which reside on human skin. Many of the microorganisms are bacteria, and most are found in the superficial layers of the epidermis and the upper parts of hair follicles. Skin microbiome is usually non-pathogenic, and either commensal or mutualistic. The benefits bacteria can offer include preventing transient pathogenic organisms from colonizing the skin surface, either by competing for nutrients, secreting chemicals against them, or stimulating the skin's immune system. Unfortunately, some resident (or native) microbes may cause skin conditions and/or diseases.

In view of the foregoing, there remains an opportunity to provide improved methods of modifying, e.g. improving, skin microbiomes. There also remains an opportunity to provide improved compositions for modifying skin microbiomes.

### SUMMARY OF THE INVENTION

A method is provided. The method is generally useful for modifying a skin microbiome. The method comprises administering a topical composition to a subject's skin. A topical composition is also provided. The topical composition may be used for the method.

The topical composition comprises a population of microorganisms, a component obtained from the population of microorganisms, or a combination thereof. The population of microorganisms is generally a Corynebacterium species. The Corynebacterium species comprises at least about 90%, optionally at least about 97%, sequence identity to a 16S rRNA sequence (SEQ ID NO: 1).

These and other objects, advantages, and features of the invention will be more fully understood and appreciated by reference to the description of the current embodiments and the drawings. Before the embodiments of the invention are explained in detail, it is to be understood that the invention is not limited to the details of operation or to the details of construction and the arrangement of the steps or components set forth in the following description or illustrated in the drawings. It is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof. Further, enumeration may be used in the description of various embodiments. Unless otherwise expressly stated, the use of enumeration should not be construed as limiting the invention to any specific order or number of components. Nor should the use of enumeration be construed as excluding from the scope of the invention any additional steps or components that might be combined with or into the enumerated steps or components.

### BRIEF DESCRIPTION OF THE DRAWINGS

FIG. 1 is a pie chart illustrating subject demographics.

FIG. 2 is a bar chart further illustrating subject demographics.

FIG. 3 is a panel illustrating bacterial diversity at each site as estimated with the Shannon Index. Each point on the graph represents the diversity score of a sample.

FIG. 4 is an ordination displaying bacterial microbial composition similarity among samples. Points represent individual microbiomes, color-coded according to site.

FIG. 5 is a box-and-whisker plot showing forehead species-level analysis of Corynebacterium (unclassified). As illustrated, relative abundance (y-axis) changes with subject age (x-axis).

FIG. 6 is a box-and-whisker plot showing forehead species-level analysis of Corynebacterium kroppenstedtii. As illustrated, relative abundance (y-axis) changes with subject age (x-axis).

FIG. 7 is a scatter chart illustrating how Corynebacterium (unclassified) and Corynebacterium kroppenstedtii are mutually exclusive.

FIG. 8 is a heat map illustrating how Corynebacterium kroppenstedtii correlates with wrinkles and age spots. As also illustrated, Corynebacterium (unclassified) generally has low to no correlation with wrinkles and age spots.

FIG. 9 is a box-and-whisker plot illustrating redness score distribution as a function of Corynebacterium kroppenstedtii relative abundance.

FIG. 10 is a series of photos illustrating a skin redness visual grading scale, ranging from low/no redness on the left (designated as 1) to higher redness on the right (designated as 5).

### DETAILED DESCRIPTION OF THE INVENTION

The method of this disclosure is useful for modifying a subject's skin microbiome. For example, the method may be used to reduce, slow, and/or prevent at least one skin condition of the subject. The method of this disclosure may also be referred to as a cosmetic method or as a treatment method.

Examples of skin conditions that may be reduced, slowed, and/or prevented via the method and/or via the topical composition of this disclosure include, but are not limited to, inflammation, redness, hyperpigmentation, wrinkling, and combinations thereof. Further skin conditions that may be reduced, slowed, and/or prevented via the method include, but are not limited to, acne, psoriasis, rosacea, eczema, vitiligo, dermatomyositis, acitinic keratosis (age spots), seborrheic keratoses, dermatitis, and combinations thereof. In various embodiments, the method and/or topical composition of this disclosure is useful for at least one of soothing and calming the subject's skin. In further embodiments, the method and/or topical composition keeps the subject's skin calm. Additional skin conditions and/or disorders are described in U.S. Pub. Nos. 2016/0271189 A1, 2017/0151291 A1, and 2017/0228514 A1, the disclosures of which are incorporated herein by reference in their entirety.

The terms “subject,” “individual,” “host,” and “patient” may be used interchangeably herein and refer to any animal subject, including: humans, laboratory animals, livestock, and household pets, typically humans. The subject can host a variety of microorganisms. The subject can have different microbiomes in various habitats on and in their body. The subject may be diagnosed or suspected of being at high risk for a disease. The subject may have a microbiome state that is contributing to a disease (i.e. dysbiosis). In some cases, the subject is not necessarily diagnosed or suspected of being at high risk for the disease.

The terms “microbiome,” “microbiota,” and “microbial habitat” may be used interchangeably herein and can refer to the ecological community of microorganisms that live on or in the subject's body. The microbiome can be comprised of commensal, symbiotic, and/or pathogenic microorganisms. Microbiomes can exist on or in many, if not most parts of the subject.

The terms “treatment” or “treating” may be used interchangeably herein. These terms can refer to an approach for obtaining beneficial or desired results including but not limited to a therapeutic benefit and/or a prophylactic benefit. A therapeutic benefit can mean eradication or amelioration of the underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The method comprises administering the topical composition to the subject's skin. In various embodiments, the topical composition is applied by hand; however, the topical composition can also be applied via an application means directly or indirectly to the skin, e.g. via an applicator, nozzle, patch, etc. In certain embodiments, the topical composition is rubbed and/or massaged on the subject's skin.

The topical composition of this disclosure may also be referred to herein simply as the composition. In addition, the composition of this disclosure may be referred to as a personal care composition, skincare composition, pharmaceutical composition, cosmetic composition, or the like. In certain embodiments, the composition is a cosmetic composition and can be used for cosmetic uses or cosmetic applications. In other embodiments, the composition is a pharmaceutical composition and can be used for pharmaceutical uses or pharmaceutical applications.

The composition may be administered as needed, daily, several times per day or in any suitable regimen such that the desired outcome is achieved. In the method, the frequency of application can depend on several factors, including the desired level of prevention, treatment, and/or effect. Generally, a regimen includes application of the composition to the skin once or twice daily to include an application in the morning and/or an application in the evening. The amount of composition applied to the skin during each application may depend on several factors including level of desired results and the specific composition.

In various embodiments, the subject is mammalian, typically a human, and can include males and females of various ages. In various embodiments, the subject is at least 18 years of age, i.e., is an adult. In certain embodiments, the subject is from about 25 to about 100, optionally about 30 to about 80, optionally about 35 to about 60, optionally about 40 to about 50, years of age. Without being bound or limited to any particular theory, it is thought that the method and composition of this disclosure are especially useful for middle-aged adults.

The composition is not limited to a particular subject or location of skin on the subject. For example, a person may apply the composition to their face, neck, arms, hands, chest, torso, legs, feet, etc., or any combination thereof. Such skin areas may be normal, dry, sensitive, oily, or combinations thereof. In various embodiments, the composition is applied to the subject's face, optionally to at least the subject's forehead. Without being bound or limited to any particular theory, it is thought that the method and composition of this disclosure are especially useful for facial skin, such as the subject's forehead, nose, cheeks, etc.

The composition comprises a population of microorganisms, a component obtained from the population of microorganisms, or a combination thereof. In certain embodiments, the composition comprises the population of microorganisms. In these or alternate embodiments, the composition comprises (or further comprises) the component obtained from the population of microorganisms.

The population of microorganisms is a Corynebacterium species. The Corynebacterium species is yet to be classified, thus it may be referred to herein simply as Corynebacterium (unclassified). That being said, the Corynebacterium species comprises at least about 90%, at least about 91%, at least about 92%, at least about 93%, at least about 94%, at least about 95%, at least about 96%, at least about 97%, at least about 98%, at least about 99%, at least about 99.5%, or 100%, sequence identity to a 16S rRNA sequence:

(SEQ ID NO: 1)
TACGTAGGGTGCGAGCGTTGTCCGGAATTACTGGGCGTAAAGGGCTCGTA
GGTGGTTTGTCGCGTCGTCTGTGAAATTCCGGGGCTTAACTCCGGGCGTG
CAGGCGATACGGGCATAACTTGAGTACTGTAGGGGTAACTGGAATTCCTG
GTGTAGCGGTGAAATGCGCAGATATCAGGAGGAACACCGATGGCGAAGGC
AGGTTACTGGGCAGTTACTGACGCTGAGGAGCGAAAGCATGGGTAGCGAA
CAGG.

The sequences described herein are generally based on targeted regions of the 16S rRNA gene, typically the V4 region (or sub-region) of the 16S rRNA gene. Methodologies using 16S rRNA sequencing are understood by those skilled in the art, and this disclosure is not limited to a particular one.

The terms “16S,” “16S ribosomal subunit,” and “16S ribosomal RNA (rRNA)” may be used interchangeably herein and can refer to a component of a small subunit (e.g., 30S) of a prokaryotic (e.g., bacteria, archaea) ribosome. Without being bound or limited to any particular theory, it is thought that the 16S rRNA is highly conserved evolutionarily among species of microorganisms. Consequently, sequencing of the 16S ribosomal subunit can be used to identify and/or compare microorganisms present in a sample (e.g., a subject's skin microbiome). The term “sequencing” as may be used herein refers to sequencing methods for determining the order of the nucleotide bases—A, T, C, G, and U—in a nucleic acid molecule (e.g., a DNA or RNA nucleic acid molecule).

The term “genome” as may be used herein, can refer to the entirety of an organism's hereditary information that is encoded in its primary DNA sequence. The genome includes both the genes and the non-coding sequences. For example, the genome may represent a microbial genome. The genetic content of the microbiome can comprise: genomic DNA, RNA, and ribosomal RNA, the epigenome, plasmids, and all other types of genetic information found in the microbes that comprise the microbiome.

The terms “nucleic acid sequence” and “nucleotide sequence” as may be used herein can refer to an oligonucleotide or polynucleotide, and fragments or portions thereof, and to DNA or RNA of genomic or synthetic origin which may be single- or double-stranded, and represent the sense or antisense strand. The nucleic acid sequence can be made up of adenine, guanine, cytosine, thymine, and uracil (A, T, C, G, and U) as well as modified versions (e.g. N6-methyladenosine, 5-methylcytosine, etc.). The terms “homology” and “homologous” as may be used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence.

The population of microorganisms can be obtained in various ways. In certain embodiments, a sample of the population is collected from facial skin, e.g. forehead skin, of one or more subjects. In various embodiments, the subjects are of younger age, e.g. less than 25 years of age, optionally less than 18 years of age. Without being bound or limited to any particular theory, it is thought that the Corynebacterium (unclassified) of this disclosure is most prevalent on the facial skin of children, teens, and young adults. The sample population can then be cultured and grown into a larger population. The population of microorganisms can be concentrated, isolated, and/or purified using methods understood in the art.

The population of microorganisms itself can be used, such that the composition may be akin to a probiotic. The term “probiotic” as used herein can mean one or more microorganisms which, when administered appropriately, can confer a health benefit on the host or subject.

In some embodiments, the component obtained from the population of microorganisms comprises a supernatant and/or a derivative thereof obtained from the population of microorganisms. In these embodiments, the population can be obtained as like described above, and then post-processed. For example, the population of microorganisms can be broken down and components/materials thereof separated, isolated, etc. These embodiments may be akin to a postbiotic.

In various embodiments, the population of microorganisms, and/or the supernatant and/or a derivative thereof obtained from the population of microorganisms, is useful for soothing and/or calming the subject's skin. It is to be appreciated that the subject(s) from which the population of microorganisms can be obtained is generally different from the subject(s) being treated via the method and/or composition of this disclosure. For example, the former subjects may have an average age lower than the average age of the latter subjects.

In general, the subject's skin includes a native population of microorganisms prior to administering the topical composition. Typically, the native population of microorganisms is different from the population of microorganisms associated with the topical composition administered to the subject's skin. For example, the native population of microorganisms may be substantially free of, or completely free of, the Corynebacterium (unclassified). Without being bound or limited to any particular theory, it is thought that the Corynebacterium (unclassified) of this disclosure is least prevalent (if present at all) on the facial skin of middle-aged adults and seniors.

In various embodiments, the native population of microorganisms comprises Corynebacterium kroppenstedtii. In certain embodiments, the Corynebacterium kroppenstedtii comprises at least about 90%, at least about 91%, at least about 92%, at least about 93%, at least about 94%, at least about 95%, at least about 96%, at least about 97%, at least about 98%, at least about 99%, at least about 99.5%, or 100%, sequence identity to a reference 16S rRNA sequence:

(SEQ ID NO: 2)
TACGTAGGGTGCGAGCGTTGTCCGGAATTACTGGGCGTAAAGAGCTCGTA
GGTGGTCTGTCGCGTCATTTGTGAAAGCCCGGGGCTTAACTCCGGGTTGG
CAGGTGATACGGGCATGACTGGAGTACTGTAGGGGAGACTGGAATTCCTG
GTGTAGCGGTGAAATGCGCAGATATCAGGAGGAACACCGGTGGCGAAGGC
GGGTCTCTGGGCAGTAACTGACGCTGAGGAGCGAAAGCATGGGTAGCGAA
CAGG.

As introduced above, the sequences described herein are generally based on targeted regions of the 16S rRNA gene, typically the V4 region (or sub-region) of the 16S rRNA gene. Methodologies using 16S rRNA sequencing are understood by those skilled in the art, and this disclosure is not limited to a particular one.

In certain embodiments, at least one of the population of microorganisms and the component obtained from the population of microorganisms is present in the topical composition in a therapeutically effective amount to reduce, slow, and/or prevent at least one skin condition of the subject.

In various embodiments, the population of microorganisms is present in an amount of from about 0.1 to about 99.9, optionally of from about 1 to about 99, optionally of from about 5 to about 95, optionally of from about 10 to about 90, optionally of from about 15 to about 85, optionally of from about 20 to about 80, optionally of from about 25 to about 75, optionally of from about 30 to about 70, optionally of from about 35 to about 65, optionally of from about 40 to about 60, optionally of from about 45 to about 55, optionally about 50, parts by weight, based on 100 parts by weight of the composition. It is contemplated that any and all values or ranges of values between those described above may also be utilized. Such amounts can be normalized to account for the inclusion of one or more additional components.

In various embodiments, the component obtained from the population of microorganisms is present in an amount of from about 0.1 to about 99.9, optionally of from about 1 to about 99, optionally of from about 5 to about 95, optionally of from about 10 to about 90, optionally of from about 15 to about 85, optionally of from about 20 to about 80, optionally of from about 25 to about 75, optionally of from about 30 to about 70, optionally of from about 35 to about 65, optionally of from about 40 to about 60, optionally of from about 45 to about 55, optionally about 50, parts by weight, based on 100 parts by weight of the composition. It is contemplated that any and all values or ranges of values between those described above may also be utilized. Such amounts can be normalized to account for the inclusion of one or more additional components.

In specific embodiments, the method is further defined as a method of treating skin inflammation or skin redness or it's reoccurrence in a subject in need thereof. In these embodiments, the method comprises administering to the subject a topical pharmaceutical composition comprising a therapeutically-effective amount of a supernatant. The supernatant is from a culture of microbes with a rRNA sequence comprising at least about 97% sequence identity to a 16S rRNA sequence from Corynebacterium (undefined; SEQ ID NO: 1; V4 region). The supernatant has the effect of decolonizing the skin of microbes comprising at least about 97% sequence identity to the 16S rRNA sequence from Corynebacterium kroppenstedtii (SEQ ID NO: 2; V4 region).

In other specific embodiments, the method is further defined as a method of treating or reducing the likelihood of development of skin inflammation or skin redness or it's reoccurrence in a subject in need thereof. In these embodiments, the method comprises administering to the subject a topical pharmaceutical composition comprising a therapeutically-effective amount of a bacterial population consisting of bacteria comprising 16S rDNA sequence at least about 97% identical to a 16S rDNA sequence present in a reference Corynebacterium (undefined; SEQ ID NO: 1; V4 region). The therapeutic composition is administered under conditions such that the bacterial population exerts an inhibitory or decolonizing effect on a pathogenic bacterium present on the skin. The pathogenic bacterium comprises at least about 97% sequence identity to the 16S rRNA sequence from Corynebacterium kroppenstedtii (SEQ ID NO: 2; V4 region).

In yet other specific embodiments, a method for diagnosing the likelihood of a subject to acquire or have clinical or sub-clinical skin inflammation is provided. In these embodiments, the (diagnostic) method uses the abundance of the bacterium present on the skin. The bacterium comprises at least about 97% sequence identity to the 16S rRNA sequence from Corynebacterium kroppenstedtii (SEQ ID NO: 2; V4 region).

In the diagnostic method, it is thought that if a subject has Corynebacterium kroppenstedtii on their skin, then the subject will have a higher likelihood of developing or having clinical or sub-clinical skin inflammation relative to a subject not having Corynebacterium kroppenstedtii on their skin. This effect may be exacerbated if the subject is of older age. In addition, if the subject has Corynebacterium kroppenstedtii on their skin, the population thereof may be quantitated and/or graded relative to other subjects, thus providing an estimate of when clinical or sub-clinical skin inflammation may occur and/or the severity thereof. Moreover, if the subject has Corynebacterium kroppenstedtii on their skin, the subject can be treated via the method and/or composition of this disclosure.

As further described in the EXAMPLES section below, it was surprisingly discovered that two mutually exclusive Corynebacterium species exist, one that is prevalent in youth (Corynebacterium (unclassified)), the other being prevalent in subjects of older age (Corynebacterium kroppenstedtii). Further, it was found that Corynebacterium kroppenstedtii is significantly associated with having skin inflammation and skin redness which are clinically important.

Surprisingly, the inventors discovered that Corynebacterium (unclassified) can be used to modify or modulate (Corynebacterium kroppenstedtii). Without being bound or limited to any particular theory, it is thought that the presence of Corynebacterium (unclassified) is preferred over having Corynebacterium kroppenstedtii being present on skin, as the latter was discovered to correlate with one or more undesirable skin conditions, such as redness. It is also thought that the presence of Corynebacterium kroppenstedtii may actually cause such skin conditions. Thus, Corynebacterium (unclassified) can be used to displace, reduce, and/or prevent Corynebacterium kroppenstedtii and the associated skin conditions thereof.

The composition may include one or more additional components as described herein, such as one or more additives. In various embodiments, the composition consists essentially of at least one of the population of microorganisms and the component obtained from the population of microorganisms. As used herein, the phrase “consisting essentially of” generally encompasses the specifically recited elements/components for a particular embodiment. Further, the phrase “consisting essentially of” generally encompasses and allows for the presence of additional or optional elements/components that do not materially impact the basic and/or novel characteristics of that particular embodiment. In certain embodiments, “consisting essentially of” allows for the presence of ≤10, ≤5, or ≤1, weight percent (wt. %) of additional or optional components based on the total weight of the composition. In other embodiments, the composition consists of at least one of the population of microorganisms and the component obtained from the population of microorganisms, as described herein.

In various embodiments, the composition further comprises at least one cosmetically acceptable carrier, excipient, additive, or combinations thereof. Suitable additives include those understood in the art, including but not limited to, moisturizers, emollients, emulsifiers, surfactants, oils, extracts, skin protectants, disinfectants, antiseptics, drugs and drug substances, analgesic compounds, antineuralgic compound, anti-oxidants, blood circulation promoters, antidepressant compounds, anti-anxiety compounds, anti-stress compounds, sunscreens, insect repellants, preservatives, exfoliants, fragrances, colors, fillers, solvents, vehicles, carriers, other types of additives known to those of skill in the art, and combinations thereof. Such additives may be utilized alone or in combination. Various optional additives are described in greater detail below.

It is to be appreciated that certain components or additives may be classified under different terms of art and just because a component or additive is classified under such a term does not mean that they are limited to that function. If utilized, the additive or additives may be present in the composition in various amounts.

The composition may include one or more moisturizers. Moisturizers may impart or restore moisture to skin. Increasing skin water content may make the skin softer and more pliable. Moisturizers may serve to mimic the action of normal skin secretions in maintaining suppleness in the skin and provide a barrier to evaporation. Skin moisturizers may include two general types: occlusives and humectants. Occlusive moisturizers form a layer on the skin which reduces the rate of evaporation. Humectants are nonocclusive hygroscopic substances which retain water and make the water available to the skin. Humectants may also function by improving the lubricity of the skin. Both occlusive and humectant moisturizers may be suitable for use in the composition of this disclosure. A moisturizer may be comprised of a single moisturizing ingredient or it may be comprised of a plurality of ingredients which may be included to serve diverse purposes such as emollients, emulsifiers, lipids, surfactants, thickeners, and preservatives. Further, a moisturizer may have both occlusive and nonocclusive properties. Water may be among the ingredients included in a moisturizer. Selection of the levels and types of moisturizers incorporated in the composition may be made without adversely affecting the stability of the composition or its in-use characteristics.

A moisturizer may include long chain C₁₂-C₂₂ fatty acids, liquid water-soluble polyols, glycerin, propylene glycol, sorbitol, polyethylene glycol, ethoxylated/propoxylated ethers of methyl glucose, ethoxylated/propoxylated ethers of lanolin alcohol, lanolin alcohol, coconut fatty acid, tallow fatty acid, nonocclusive liquid water-soluble polyols, aloe vera gel, aloe vera gel condensed, aloe vera gel freeze-dried powder, aloe vera gel oil extract, amino acids, amniotic fluid, avocadin, calcium protein complex, cashew oil, chia oil, chitin, chitosan, chitosan PCA, cholesteric esters, chondroitin sulfate, collagen, collagen amino acids, copper protein complex, dioctyl maleate, dipentaerythritol fatty acid ester, elastin, ethyl panthenol, evening primrose oil, glycereth-12, glycosphingo lipids, hyaluronic acid, hybrid safflower oil, hydrogenated polyisobutene, hydrolyzed collagen, hydrolyzed elastin, hydrolyzed fibronectin, hydrolyzed mucopolysaccharides, hydrolyzed silk, hydrolyzed wheat protein, jojoba esters, keratin amino acids, kiwi fruit extract, lactamide MEA, liposomes, live yeast cell derivative liposome, marina polyaminosaccharide, mineral oil, mink oil ethyl ether, mucopolysaccharides, mucopolysaccharides, palmetto extract, pantethine, paraffin, PEG-4, PEG-6, PEG-8, PEG-12, PEG-100 stearate, perfluoropolymethyl-isopropyl ether, petrolatum, petroleum wax, pistachio oil, placenta extract, plankton extract, polyamino sugar condensate, polybutene, polyglyceryl methacrylate, polypentaerythrityl tetralaurate, PPG-10 butanediol, PPG-20 methyl glucose ether distearate, royal jelly extract, saccharide isomerate, selenium protein complex, serum albumin, sodium hyaluronate dimethylsilanol, sodium lactate methylsilonol, sodium mannuronate methylsilanol, soluble collagen, super oxide dismutase, super oxide dismutase liposome, tissue extract, tocopheryl linoleate, lipophylic moisturizers such as lysolecithin, lecithin, cholesterol, cholesterol esters, sphingolipids, or ceramides, low molecular moisturizer such as serine, glutamine, sorbitol, mannitol, glycerin, sodium pyrrolidone-carboxylate, 1,3-butylene glycol, propylene glycol, lactic acid, or lactic acid salts, high molecular moisturizers such as hyaluronic acid, sodium hyaluronate, elastin, alginic acid, mucopolysaccharides, polyethylene glycol, polyaspartic acid salts, or water soluble chitin, hydrocarbon oils, hydrocarbon waxes, silicones, fatty acid derivatives, cholesterol, cholesterol derivatives, di- and tri-glycerides, vegetable oils, vegetable oil derivatives, liquid nondigestible oils, blends of liquid digestible or nondigestible oils with solid polyol polyesters, acetoglyceride esters, alkyl esters, alkenyl esters, lanolin and its derivatives, milk tri-glycerides, wax esters, beeswax derivatives, sterols, phospholipids, or any other moisturizer ingredient.

An occlusive moisturizer may be petrolatum, paraffin, waxes, greases, mineral oil, beeswax, lanolin and oil-soluble lanolin derivatives, saturated and unsaturated fatty alcohols such as behenyl alcohol, squalene, various animal and vegetable oils such as almond oil, apricot oil, apricot pit oil, avocado oil, cade oil, castor oil, cinnamon oil, corn oil, cottonseed oil, evening primrose oil, grape oil, grape seed oil, hazelnut oil, jojoba oil, linseed oil, liver oil, macadamia nut oil, mink oil, neetsfoot oil, olive oil, palm kernel oil, palm nut oil, palm oil, peach pit oil, peanut oil, pine oil, pistachio nut oil, poppyseed oil, rapeseed oil, rice bran oil, rice germ oil, safflower oil, sasanqua oil, sesame oil, sesame seed oil, soybean oil, sunflower oil, sunflower seed oil, tsubaki oil, walnut oil, wheat germ oil, wheat germ oil, teaseed oil, triglycerine, glycerine trioctanate, glycerine triisopalmitate, cacao fat, beef tallow, sheep fat, hog fat, horse fat, hydrogenated oil, hydrogenated castor oil, Japanese wax, shea butter, beeswax, candelilla wax, cotton wax, carnauba wax, bayberry wax, tree wax, spermaceti, montan wax, bran wax, lanolin, reduced lanolin, hard lanolin, kapok wax, sugarcane wax, jojoba wax, shellac wax, or any other moisturizer exhibiting occlusive properties.

A moisturizer may include agents that mimic natural ingredients and function as botanicals, including vitamins, hydroxy acids, and retinoids. Vitamins may include vitamin A, retinol, retinol palmitate, inositol, pyridoxine chlorate, benzyl nicotinate, nicotinamide, dlα-tocopheryl nicotine, magnesium ascorbyl phosphate, vitamin D₂ (ergocalciferol), dlα-tocopherol, potassium dl-α-tocopherol-2-L-ascorbic diester, dl-α-tocopheryl acetate, pantothenic acid, biotin, or any other vitamin. Some ingredients that may reduce the severity of dry skin are alpha hydroxy acids (AHA) and beta hydroxy acids (BHA), including their salts, as well as retinoids. The hydroxy acids are classified according to the number of carboxylic acids on their configuration. Monocarboxylic acids are glycolic, lactic, and mandelic acids. Dicarboxylic acids include malic and tartaric acids. Tricarboxylic acids embody citric acid found in citrus fruits. The BHAs encompass mostly salicylic acid and its derivatives. AHAs have been shown to exfoliate. Thus, they are useful in hyperkeratotic conditions. They act as humectants and have a normalizing effect on the stratum corneum, increasing its plasticity and flexibility. Other ingredients of a moisturizer may include elastin, lecithin, sodium hyaluronate, sodium passive cutaneous anaphylaxis, ceramides, naturally occurring skin lipids and sterols, artificial or natural oils, humectants, emollients, emulsifiers, preservatives, lubricants, greases, natural moisturizing factors (NMF) including low molecular weight substances such as ammonia, amino acids, glucosamine, creatinine, citrate and ionic solutions such as sodium, potassium, chloride, phosphate, calcium and magnesium, sodium pyrrolidone carboxylic acid, hexadecyl, myristyl, isodecyl, or isopropyl esters of adipic, lactic, oleic, stearic, isostearic, myristic and linoleic acids, and their corresponding alcohol esters, sodium isostearoyl-2-lactylate and sodium capryl lactylate, glycerin, polyethylene glycol, propylene glycol, sorbitol, polyethylene glycol and propylene glycol ethers of methyl glucose, polyethylene glycol and propylene glycol ethers of lanolin alcohol, lactic acid, L-proline, and other free fatty acids, coconut fatty acid, tallow fatty acid, nonocclusive liquid water-soluble polyols and the essential amino acid compounds found naturally in the skin, and stearic and lauric acids.

The composition may include one or more emollients. Emollients may smooth roughened skin, change the skin's appearance, lubricate, replace natural skin lipids, and provide occlusion. Emollients may be composed of water-in-oil emulsions. An emollient may make something soft or supple, and may also sooth the skin or mucous membrane. Emollients, such as lanolin, shea butter, or petrolatum may act as a barrier (occlusion effect) against loss of water and also as a softener of stratum corneum. Other emollients may be oil-water emulsions of varying composition and may include several esters and oils such as octyl dodecanol, hexyl decanol, oleyl alcohol, decyl oleate, isopropyl stearate, isopropyl palmitate, isopropyl myristate, hexyl laureate, and dioctyl cyclohexane. Further, emollients may include long-chain acylglutamic acid cholesteryl esters, cholesteryl hydroxystearate, 12-hydroxystearic acid, stearic acid, rhodinic acid, lanolin fatty acid cholesteryl ester, petrolatum, cocoa butter, esters of fatty acids, glycerin mono-, di-, and tri-esters, epidermal and sebaceous hydrocarbons such as cholesterol, cholesterol esters, squalane, silicone oils and gums, mineral oil, lanolin and derivatives, castor oil, almond oil, oleyl oleate, or any other emollient ingredient.

The composition may include one or more emulsifiers. An emulsifier may be a substance that is capable of lowering the interfacial tension between an oil and an aqueous phase and, thus, may aid the dispersal of oil (in the case of oil-in-water emulsions) and water (in the case of water-in-oil emulsions), respectively, into droplets of a small size and help to maintain the particles in a dispersed state. Emulsifiers may be generally classified as i) proteins or carbohydrate polymers, which act by coating the surface of the dispersed fat or oil particles, thus preventing them from coalescing; such emulsifiers are sometimes also called protective colloids, and ii) long-chain alcohols and fatty acids, which are able to reduce the surface tension at the interface of the suspended particles because of the solubility properties of their molecules. Soaps behave in this manner when they exert cleaning action by emulsifying the oily components of soils.

The composition may include one or more surfactants. Surfactants may be detergent, soap base, sodium laurate, sodium palmitate, or any other fatty acid soap, sodium laurosulfate, potassium laurosulfate, or any other higher alkyl sulfate ester salt, POE laurosulfate triethanol amine, sodium POE laurosulfate, or any other alkyl ester sulfate ester salt, sodium lauroylsarcosine or any other N-acylsarcosine acid, sodium N-myristyl-N-methyltaurine, sodium N-cocoyl-N-methyl taurate, sodium laurylmethyl taurate, or any other higher fatty acid amide sulfonate, sodium POE oleyl ether phosphate, POE stearyl ether phosphate, or any other phosphate ester salt, sodium di-2-ethylhexyl-sulfosuccinate, sodium monolauroylmonoethanol amide polyoxyethylene sulfosuccinate, sodium laurylpoly-propylene glycol sulfosuccinate, or any other sulfosuccinate, linear sodium dedecylbenzensulfonate, linear dodecylbenzensulfonate triethanol amine, linear dodecyl benzensulfate, or any other alkylbenzensulfonate, sodium N-lauroylglutamate, disodium N-stearoylglutamate, monosodium N-myristoyl-L-glutamate, or any other N-acylglutamate, sodium hydrogenated castor oil fatty acid glycine sulfate or any other higher fatty acid ester sulfate ester salt, Turkey red oil or any other sulfated oil, POE alkyl ether carboxylic acid, POE alkylaryl ether carboxylate, α-olefinsulfates, higher fatty acid ester sulfonates, secondary alcohol sulfate ester salts, higher fatty acid alkylolamide sulfate ester salts, sodium lauroyl monoethanolamide succinate, N-palmitoyl asparaginate ditriethanol amine, sodium caseine, or any other anionic surfactant, stearyl trimethyl ammonium chloride, lauryl trimethyl ammonium chloride, or any other alkyl trimethyl ammonium salt, distearyldimethyl ammonium chloride, dialkyldimethyl ammonium chloride salts, poly(N,N′-dimethyl-3,5-methylenepiperidinium)chloride, cetylpyridinium chloride or any other alkyl pyridinium salt, alkyl quaternary ammonium salts, alkyl dimethylbenzyl ammonium salts, alkyl isoquinolinium salts, dialkyl morphonium salts, POE alkyl amines, alkyl amine salts, polyamine fatty acid derivatives, amyl alcohol fatty acid derivatives, benzalkonium chloride, benzethonium chloride, or any other cationic surfactant, sodium 2-undecyl-N,N,N-(hydroxyethylcarboxymethyl)-2-imidazoline, 2-cocoyl-2-imidazoliniumhydroxide-1-carboxyethyloxy-2-sodium salt, or any other imidazoline family bipolar surfactant, 2-heptadecyl-N-carboxymethyl-N-hydroxyethylim idazolinium betaine, lauryldimethyl-aminoacetate betaine, alkyl betaine, amide betaine, sulfo betaine, or any other betaine family surfactant, or any other bipolar surfactant, sorbitan monooleate, sorbitan monoisostearate, sorbitan monolaurate, sorbitan monopalmitate, sorbitan monostearate, sorbitan sesquioleate, sorbitan trioleate, diglyceryl sorbitan pentaoctanoate, diglyceryl sorbitan tetraoctanoate, or any other sorbitan fatty acid ester, glycerin mono cotton seed oil fatty acid, glycerin monoerucate, glycerin sesquioleate, glycerin monostearate, glycerin α,α-oleate pyroglutamate, monostearate glycerin malic acid or any other glycerin or polyglycerin fatty acid, propylene glycol monostearate or any other propylene glycol fatty acid ester, hydrogenated castor oil derivatives, glycerin alkyl ethers, polyoxyethylene methylpolysiloxane copolymers, or any other lyophilic nonionic surfactant, POE sorbitan monooleate, PO-sorbitan monostearate, POE-sorbitan monooleate, POE-sorbitan tetraoleate, or any other POE sorbitan fatty acid ester, POE-sorbite monolaurate, POE-sorbitan monooleate, POE-sorbite pentaoleate, POE-sorbitan monostearate, or any other POE sorbitan fatty acid ester, POE-glycerin monostearate, POE-glycerin monoisostearate, POE-glycerin triisostearate, or any other POE glycerin fatty acid ester, POE monooleate, POE distearate, POE monodioleate, distearate ethylene glycol, or any other POE fatty acid ester, POE lauryl ethers, POE oleyl ethers, POE stearyl ethers, POE behenyl ethers, POE2-octyldodecyl ethers, POE cholestanol ethers, or any other POE alkyl ether, POE octyl phenyl ethers, POE nonyl phenyl ethers, POE dinonyl phenyl ethers, or any other POE alkyl phenyl ether, Pluronic or any other poloxamer, POE.POP cetyl ethers, POE.POP-2-decyltetradecyl ethers, POE.POP monobutyl ethers, POE.POP hydrated lanolin, POE.POP glycerin ethers, or any other POE-POP alkyl ether, Tetronic or any other tetra-POE.tetra-POP ethylene diamine condensation product, POE castor oil, POE hydrogenated castor oil, POE hydrogenated castor oil monoisostearate, POE hydrogenated castor oil triisostearate, POE hydrogenated castor oil monopyroglutamate monoisostearate diester, POE hydrogenated castor oil maleic acid or any other POE castor oil hydrogenated castor oil derivative, POE sorbitan beeswax or any other POE beeswax lanolin derivative, coconut oil fatty acid diethanolamide, laurate monoethanolamide, fatty acid isopropanolamide, or any other alkanolamide, POE propylene glycol fatty acid esters, POE alkylamines, POE fatty acid amides, sucrose fatty acid esters, POE nonylphenyl formaldehyde condensation products, alkylethoxydimethylamineoxide, trioleylphosphoric acid, or any other hydrophilic nonionic surfactant, or any other surfactant.

The composition may include one or more oils. Oils may act as penetrating transdermal carriers that penetrate the skin the quickly and aid in transport of other components present in the composition of the present invention. Examples of oils that may be utilized include almond oil, anise oil, apricot kernel oil, apricot oil, avocado oil, balm mint oil, basil oil, bee balm oil, bergamot, bergamot oil, birch oil, bitter almond oil, bitter orange oil, caraway oil, cardamom oil, castor oil, cedarwood oil, cinnamon oil, clay oil, clove oil, cloveleaf oil, coconut oil, fractionated coconut oil, cottonseed oil, cypress oil, eucalyptus oil, evening primrose oil, fennel oil, gardenia oil, geranium oil, ginger oil, grapefruit oil, grape seed oil, hazelnut oil, hops oil, hyptis oil, indigo bush oil, jasmine oil, jojoba oil, juniper oil, kiwi oil, kukui nut oil, laurel oil, lavender oil, lemon oil, lemongrass oil, linden oil, linseed oil, lovage oil, macadamia nut oil, maize oil, matricaria oil, musk rose oil, neroli oil, nutmeg oil, olibanum, olive oil, orange flower oil, orange oil, palm oil, patchouli oil, peach kernel oil, peanut oil, pecan oil, pennyroyal oil, peppermint oil, persic oil, pine oil, pine tar oil, poppy-seed oil, rapeseed oil, rose oil, rose hips oil, rosemary oil, rue oil, sage oil, sambucus oil, sandalwood oil, sassafras oil, sesame oil, silver fir oil, soybean oil, spearmint oil, sunflower oil, sweet almond oil, sweet marjoram oil, sweet violet oil, tar oil, tea tree oil, thyme oil, wheat germ oil, wild mint oil, yarrow oil, ylang ylang oil, walnut oil, tall oil, thistle seed oil, hydrogenated vegetable oils, or any other suitable oil.

The composition may include essential oils, extracts, and combination thereof. Essential oils are typically concentrated liquids containing volatile aroma compounds from plants. Essential oils may also be referred to as volatile oils, ethereal oils, aetherolea, or simply as the oil of the plant from which they were extracted. An oil is typically “essential” in the sense that it contains the essence of the plant's fragrance—the characteristic fragrance of the plant from which it is derived.

Various sections or parts of plants can be used to obtain the essential oils and extracts, such as bark, berries, flowers, fruits, leaves, peels, resins, rhizomes, roots, seeds, and/or woods. Essential oils can be obtained by a number of processes, such as by distillation (e.g. using steam), expression, solvent extraction, absolute oil extraction, resin tapping, and/or cold pressing.

A number of different extraction methods may be used to obtain extracts suitable for this disclosure. These extraction methods include, but are not limited to, the extraction methods disclosed in U.S. Pat. No. 7,897,184 to Rana et al., which is hereby incorporated by reference in its entirety. While extraction solvents described specifically mention ethanol, it should be understood that other alcohols such as, but not limited to, isopropyl alcohol, ethyl alcohol, and/or methyl alcohol may be used in addition to or as an alternative to ethanol. Exemplary alcoholic solvents include, but are not limited to, C₁ to C₄ alcohols, such as methanol, ethanol, propanol, isopropanol, and butanol; hydro-alcohols or mixtures of alcohol and water, including hydro-ethanol; polyhydric alcohols such as propylene glycol and butylene glycol; and fatty alcohols. Any of these alcoholic solvents may be used. Other solvents such as, but not limited to, acetone may also be used as an extraction solvent. Solvent-water blends, e.g. alcohol-water and/or acetone-water blends, of any ratio, may also be used. In various embodiments, the solvent is one in which the resulting extract and/or a subsequent form thereof (e.g. extract powder) is suitable for ingestion. For example, the solvent is water or ethanol.

In one example, the extracts can be obtained using an organic solvent extraction technique. In another example, solvent sequential fractionation can be used to obtain the extracts. Total hydro-ethanolic extraction techniques can also be used to obtain the extracts. Generally, this is referred to as a lump-sum extraction. The extract generated in the process will contain a broad variety of phytochemicals present in the extracted material including fat and water-soluble phytochemicals. Following collection of the extract solution, the solvent will be evaporated, resulting in the extract.

Total ethanol extraction may also be used. This technique uses ethanol as the solvent. This extraction technique generates an extract that may include fat soluble and/or lipophilic compounds in addition to water-soluble compounds. Total methanol extraction may also be used in a similar manner with similar results.

Another example of an extraction technique that can be used to obtain the extracts is supercritical fluid carbon dioxide extraction (SFE). In this extraction procedure, the material to be extracted is not exposed to any organic solvents. Rather, the extraction solvent is carbon dioxide (CO₂), with or without a modifier, in super-critical conditions (e.g. >31.3° C. and >73.8 bar). Those of skill in the art will appreciate that temperature and pressure conditions can be varied to obtain the best yield of extract. This technique generates an extract of fat soluble and/or lipophilic compounds, similar to total hexane and ethyl acetate extraction techniques, which may also be used.

Each of the extraction methods above also may include and/or be utilized in combination with one or more additional processing steps understood in the art. For example, plant material may be comminuted, smashed, ground, etc. There also may be one or more filtration steps to remove, for example, cellulosic/fibrous or other solid materials. There also may be one or more purification steps to remove, for example, certain constituents and/or contaminants. Such purification may be accomplished, for example, by distillation, evaporation, centrifugation, etc. There also may be one or more concentration and/or drying steps to remove water and/or other volatiles, e.g. alcohol, lighter compounds, VOCs, etc. Moreover, acids and/or bases may be added to adjust pH or neutralize. Depending on the desired form of the final/end extract, one can also utilize various additional steps understood in the art, such as screening, pressing, milling, grinding, mixing, dispersing, etc. It is to be appreciated that combinations of these additional processing steps in duplicative and/or different orders is also contemplated. It is also to be appreciated that the method of this disclosure is not limited to a particular method of obtaining essential oil or extract, if utilized in or as the scented constituent.

Examples of essential oils that may be used include, but are not limited to, Agar oil or Oodh oil, Agarwood oil, Ajwain oil, Allspice oil, Angelica oil, Anise oil, Apricot kernel oil, Asafoetida oil, Balm Mint oil, Balsam Copaiba oil, Balsam oil, Basil oil, Bay Laurel oil, Bay oil, Benzoin oil, Bergamot oil, Birch oil, Black Pepper oil, Blood Orange oil, Buchu oil, Calamodin oil or Calamansi Oil, Calamus oil, Camphor oil, Cannabis oil, Caraway seed oil, Cardamom seed oil, Carrot oil, Carrot seed oil, Cassia oil, Catnip oil, Cedar oil or Cedarwood oil, Celery oil, Centella oil, Chamomile oil, Cinnamon oil, Citron oil, Citronella oil, Clary Sage oil, Clove oil, Coconut oil, Coffee oil, Copaiba oil, Coriander oil, Costmary oil or Bible leaf oil, Costus root oil, Cranberry seed oil, Cubeb oil, Cumin oil or Black seed oil, Curry leaf oil, Cypress oil, Cypriol oil, Davana oil, Dill oil, Elecampane oil, Elemi oil, Eucalyptus oil, Fennel oil, Fenugreek oil, Fir needle oil, Flax oil, Frankincense oil, Galangal oil, Galbanum oil, Geranium oil, Ginger oil, Goldenrod oil, Grapefruit oil, Guava oil, Helichrysum oil, Hickory nut oil, Hop oil, Horseradish oil, Hyssop oil, Jasmine oil, Juniper berry oil, Labdanum oil, Laurus nobilis oil, Lavender oil, Ledum oil, Lemon oil, Lemongrass oil, Lime oil, Linalool oil, Litsea cubeba oil, Lotus oil, Magnolia oil, Mandarin oil, Manuka oil, Marjoram oil, Melaleuca oil, Melissa oil or Lemon balm oil, Mentha arvensis oil, Mint oil, Moringa oil, Mountain Savory oil, Mugwort oil, Mustard oil, Myrrh oil, Myrtle oil, Neem oil, Neroli oil, Niaouli oil, Nutmeg oil, Olive oil, Orange oil, Oregano oil, Orris oil, Palmarosa oil, Palo Santo oil, Parsley oil, Patchouli oil, Pelargonium oil, Pennyroyal oil, Peppermint oil, Perilla oil, Petitgrain oil, Pine oil, Plumeria oil, Radiata oil, Ravensara oil, Red Cedar oil, Roman Chamomile oil, Rose oil, Rosehip oil, Rosemary oil, Rosewood oil, Sage oil, Sandalwood oil, Sassafras oil, Savory oil or Satureja oil, Schisandra oil, Spearmint oil, Spikenard oil, Spruce oil, Star Anise oil, Sweet Annie oil, Tangerine oil, Tansy oil, Tarragon oil, Tea tree oil, Thyme oil, Turmeric oil, Valerian oil, Verbena oil, Vetiver oil or Khus oil, Warionia oil, Wintergreen oil, Wormwood oil, Yarrow oil, Ylang-ylang oil, Zedoary oil, and combinations thereof.

Examples of extracts that may be used include, but are not limited to, acacia extract, alfalfa extract, algae extract, almond extract, aloe barbadensis extract, aloe extract, althea extract, anise extract, apple extract, apricot extract, arnica extract, arnica montana extract, artichoke extract, asafoetida extract, avocado extract, azulene extract, balm mint extract, balm mint extract, bamboo extract, banana extract, barley extract, bearberry extract, bee pollen extract, beet extract, bilberry extract, birch leaf extract, black cohosh extract, black currant extract, black walnut extract, blackberry extract, blackberry leaf extract, bladderwrack extract, blueberry extract, borage extract, botanical extracts, buckwheat extract, burdock extract, burnet extract, butcher's broom extract, calendula extract, camellia sinensis extract, camomile extract, caper extract, capsicum frutescens extract, carrageenan extract, carrot extract, cherimoya extract, cherry bark extract, cherry extract, cinchona extract, cinquefoil extract, citrus blossom extract, clover blossom extract, coltsfoot extract, coneflower extract, corn silk extract, cornflower extract, cornfrey extract, couch grass extract, crataegus extract, crataegus monogina extract, cucumber extract, cypress extract, dandelion extract, dog rose hips extract, elder flower extract, eleuterococcus extract, elm bark extract, English oak extract, eucalyptus extract, everlasting extract, fennel extract, fenugreek extract, fern extract, fig extract, gardenia extract, garlic extract, gerrtian extract, ginger extract, gingko biloba extract, ginkgo extract, ginko extract, ginseng extract, grape extract, grape leaf extract, grape seed extract, grape skin extract, goji berry extract, guarana extract, Hawaiian ginger extract, hayflower extract, helichrysum extract, henna extract, hibiscus extract, hops extract, horsechestnut extract, horsetail extract, hypericum extract, indian cress extract, ivy extract, Job's tears extract, jojoba oil, jujube extract, juniper extract, juniperus communis extract, Karite extract, kelp extract, kiwi extract, krameria triandra extract, lady's mantle extract, laminaria digitata extract, laminaria extract, lavender extract, lemon balm extract, lemon extract, lemon peel extract, lettuce extract, licorice extract, linden extract, lithospermum officinale extract, madder extract, mallow extract, mango extract, marshmallow extract, matricaria extract, melon extract, milfoil extract, mimosa tenuiflora bark extract, mistletoe extract, monk fruit extract, mushroom extract, myrrh extract, nettle extract, oak root extract, oat extract, oleoresin, onion extract, orange blossom extract, orange flowers extract, oyster shell extract, pansy extract, parsley extract, papaya extract, passion fruit extract, peach extract, pellitory extract, pennyroyal extract, peppermint extract, periwinkle extract, pine needle extract, pineapple extract, pistachio extract, plantain extract, pollen extract, quillaja saponaria extract, quince seed extract, raspberry extract, rauwolfia extract, restharrow extract, rhatany extract, rhubarb root extract, rice bran extract, rose hips extract, rosemary extract, sage extract, sambucus extract, sanguinaria root extract, saponaria extract, sea weed extract, soapwort extract, soy extract, spearmint extract, St. John's wort extract, stinging nettle extract, strawberry extract, sugar cane extract, sunflower extract, sweet clover extract, tea extract, thistle extract, thyme extract, tomato extract, tormentill extract, valerian extract, vanilla extract, violet extract, walnut extract, watercress extract, wheat bran extract, wheat germ extract, white nettle extract, white oak bark extract, white willow bark extract, wild indigo extract, willow bark extract, witch hazel extract, yarrow extract, and combinations thereof.

The composition may include one or more skin protectants. Examples of suitable skin protectants that may be utilized include allantoin, aloe vera gel, anise extract, avocado oil unsaponifiables, carboxymethyl chitin, chondroitin sulfate, collagen, collagen amino acids, embryo extract, glyceryl ricinoleate, hydrolyzed animal elastin, hydrolyzed milk protein, hydrolyzed vegetable protein, linoleic acid (and) linolenic acid (and) arachidonic acid, liposomes, perfluoropolymethyl-isopropyl ether, plankton extract, and spine marrow extract.

The composition may include one or more drug substances. Incorporation of a drug substance in the composition may be useful for the prevention or treatment of various skin disorders or to deliver drug substances to the skin which are advantageously administered topically for percutaneous absorption. A drug substance may be any compound or mixture thereof that may produce a beneficial effect on the human to whom the drug substance has been given. Drug substances may be any physiologically or pharmacologically substance that produces a localized or systemic effect in mammals including humans. Examples of suitable drug substances that may be utilized include anti-inflammatory compounds, analgesics, tranquilizers, cardiac glycosides, narcotic antagonists, antiparkinsonism agents, antidepressants, antineoplastic agents, immunosuppressants, antiviral agents, antibiotic agents, appetite suppressants, antiemitics, antihistamines, antimigraine agents, coronary, cerebral or peripheral vasodilators, antianginals, calcium channel blockers, hormonal agents, contraceptive agents, antithrombotic agents, antihypertensive agents, chemical dependency drugs, local anesthetics, corticosteroids, dermatological agents and the like, vitamins like vitamin A such as all-trans retinol, retinol acetate, retinol palmitate, retinol propionate, betacarotene, halibut-liver oil, shark-liver oil, vitamin B₁ such as thiamine hydrochloride, benfotiamine, bisbentiamine, bisbutiamine, bisibutiamine, betoiamine hydrochloride, cetotiamine hydrochloride, cocarboxylase, cycotiamine, fursultiamine, vitamin B₂ such as riboflavine, riboflavine tetrabutyrate, flavine adenine dinucleotide, vitamin B₆, vitamin B₁₂ such as cobalamins, B₁₂ TAM, cobamamide, cyanocobalamin, mecobalamin, other vitamins of the B group, vitamin C such as ascorbic acid, vitamin D such as ergocalciferol (vitamin D₂), cholecalciferol (vitamin D₃), calcifediol, calcitriol, alfacalcitriol, dihydrotachysterol, alfacalcidol, calcifediol, calcitriol, cholecalciferol, cod-liver oil, dihydrotachysterol, ergocalciferol, vitamin E, alpha tocopherols, tocopheryl nicotinate, tocopherylquinone, wheat-germ oil, vitamin K such as phytomenadione, menadiol sodium diphosphate, menadione, vitamin P, sucrose sulfate esters such as sucralfate, sucrose octasulfate and salts, esters and complexes thereof, antibacterials such as phenoxyethanol, or any other drug substance.

The composition may include one or more analgesic compounds. Examples of suitable analgesic compounds that may be utilized include aloe vera, MSM, emu oil, menthol, glucosamine, chondroitin, a capsaicinoid, arnica extract, coriander oil, Roman chamomile oil, willow bark extract, feverfew extract, St. John's wort extract, kava kava extract, nettle leaf, acetylsalicylic acid, Bala, black cohosh, black snakeroot, bugbane, squawroot, bupleurum, calendula, camphor, cayenne, devil's claw root, evening primrose oil, ginger, gotu kola, gingkgo, juniper, lavender oil, licorice, marjoram, meadow sweet, menthol, passion flower, quercetin, salicinum, wild yam, wintergreen, wood betony, wormwood, or any other analgesic.

The composition may include one or more anti-inflammatory compounds. Examples of suitable anti-inflammatory compounds that may be utilized include aloe vera, MSM, emu oil, chondroitin, glucosamine, a capsaicinoid, arnica extract, grape seed extract, coriander oil, marigold extract, nettle leaf extract, Roman chamomile oil, blue-bottle extract, St. John's wort, willow bark extract, witch hazel extract, feverfew extract, barley grass, black cohosh, black snakeroot, bugbane, squawroot, Boswellia, borage, bromelain, burdock, calendula, cayenne, dandelion, devil's claw root, DHEA (dehydroepiandosterone), Echinacea, elderflower, evening primrose oil, flaxseed, ginkgo, ginger, ginseng, Hawthorne, kaempferol, licorice, life root, golden Senecio, squaw weed, golden groundsel, cocash weed, coughweed, ragwort, golden ragwort, grundy swallow, linden, marjoram, meadow sweet, NDGA, neem, Padma 28, quercetin, shea butter, turmeric, wild yam, wormwood, yucca, bisabolol, sucralfate, LIPACIDE, gauaiazulene, essential fatty acids, poly-unsaturated fatty acid derivatives from plant seed oils and other vegetable sources, or any other anti-inflammatory. Essential fatty acids (EFAs) may include omega-3 and omega-6 fatty acids such as linolenic acid and alpha linolenic acid. In addition, any known herbs or various compounds that contain EFAs may be included in the composition. Examples of such herbs include flaxseed and evening primrose oil.

The composition may include one or more antineuralgic compounds. Compounds having antineuralgic effects generally provide relief of pain or discomfort along a course of a nerve or in an area of distribution of the nerve. Suitable antineuralgics that may be utilized include a capsaicinoid, Roman chamomile oil, coriander oil, or any other antineuralgic compound.

The composition may include one or more anti-oxidants. Compounds having anti-oxidant activity generally prevent damage or deterioration of tissue. Examples of suitable anti-oxidants that may be utilized include chondroitin, ascorbic acid, vitamin C, cocoa butter, grape seed extract, St. John's wort extract, coriander oil, cysteine, barley grass, bilberry, Echinacea, garlic, ginger, ginkgo, ginseng, grape seed proanthocyanidin extract, green tea, Hawthorne, lemon balm, milk thistle, oregano, peppermint, pomegranate juice, purslane, pycnogenol, red wine, rosemary, schizandra, wuweizi, wurenchun, trilinolein, sanchi, tartaric acid, turmeric, α-tocopherol or any other tocopherol, dibutylhydroxytoluene butylhydroxyanisole, or any other anti-oxidant.

The composition may include one or more blood circulation promoters. Blood circulation promoters generally provide increased blood circulation to an area to which the composition is applied. Examples of suitable blood circulation promoters that may be utilized include MSM (methylsulfonylmethane), arnica extract, Roman chamomile oil, nettle extract, marigold extract, grape seed extract, blue-bottle extract, coriander oil, lime tree extract, marigold extract, feverfew extract, St. John's wort extract, witch hazel extract, arjuna, Bala, benzoin, bilberry, black pepper, blue gum eucalyptus, blue vervain, borneol, butcher's broom, cayenne, cypress, geranium, ginger, ginkgo, grape seed proanthocyanidin extract, Hawthorne, L-arginine, lemon, lemon grass, linden flowers, niaouli, oat straw, orange blossom, passion flower, Peru balsam, pine, prickly ash bark, rose oils, rosemary, Spanish sage, spruce, Tien Chi ginseng, thyme, violet, white birch, yohimbe, or any other blood circulation promoter.

The composition may include one or more compounds having antidepressant, anti-anxiety, or anti-stress activity. Examples of suitable antidepressant, anti-anxiety, or anti-stress compounds that may be utilized include MSM, kava kava extract, Roman chamomile extract, feverfew extract, St. John's wort extract, bee pollen, bergamot, black cohosh, black horehound, bugleweed, California poppy, clary sage, cowslip, damiana, DHEA (dehydroepiandrosterone), geranium, ginseng, gotu kola, grapefruit, hyssop, Jamaican dogwood, lady's slipper, lavender, lemon balm, licorice, linden, lobelia, mate, mistletoe, motherwort, mugwort, oat straw, passion flower, peppermint, rosemary, skullcap, valerian root, vervain, wild lettuce, wood betony, or any other antidepressant, anti-anxiety, or anti-stress compound.

The composition may further include any pain relieving, anti-inflammatory, anti-oxidant, blood circulation promoter, anti-depressant, anti-anxiety, or anti-stress type of herb. Examples of suitable herbs that may be utilized include arjuna, Bala, barley grass, bee pollen, benzoin, bergamot, bilberry, black cohosh, black horehound, black pepper, blue gum eucalyptus, blue vervain, borage, borneol, Boswellia, bromelain, bugleweed, bupleurem, burdock, butcher's broom, California poppy, camphor, cayenne, clary sage, cocash weed, cowslip, coughweed, cypress, damiana, dandelion, devil's claw root, DHEA, echinacea, elderflower, evening primrose oil, flaxseed, garlic, geranium, ginger, ginkgo, ginseng, golden groundsel, golden ragwort, golden Senecio, gotu kola, grapefruit, grape seed proanthocyanidin extract, green tea, grundy swallow, Hawthorne, heather, hyssop, Jamaican dogwood, juniper, kaempferol, L-arginine, lady's slipper, lavender, lemon, lemon balm, lemon grass, licorice, life root, linden, lobelia, marjoram, mate, meadow sweet, milk thistle, mistletoe, motherwort, mugwort, NDGA (nordihydroguaiaretic acid), neem, niaouli, oat straw, orange blossom, oregano, Padma 28, passion flower, peppermint, Peru balsam, pine, pomegranate juice, prickly ash bark, purslane, pycnogenol, quercetin, ragwort, red wine, rose oils, rosemary, salicinum, schizandra, sharp sorrel, skullcap, Spanish sage, spruce, squaw weed, Tien Chi ginseng, thyme, trilinolein, turmeric, valerian root, vervain, violet, white birch, wild lettuce, wild yam, wintergreen, wood betony, wormwood, yohimbe, yucca, or any other pain relieving, anti-inflammatory, anti-oxidant, blood circulation promoting, anti-depressant, anti-anxiety, or anti-stress type of herb.

The composition may include one or more medicinal extracts. The medicinal extracts may have various medicinal effects. Examples of suitable medicinal extracts that may be utilized include aloe extract, candock extract, carrot extract, cinchona extract, clove extract, common fennel extract, cornflower extract, creeping saxifrage extract, cucumber extract, dishcloth gourd extract, eucalyptus extract, field horsetail extract, hamamelis extract, herbaceous peony extract, horse chestnut extract, Houttuynia cordate extract, iris rhizome extract, lemon extract, licorice root extract, Lithospermum erythrorhizon extract, melilot extract, melissa extract, mulberry extract, peach extract, peach leaf extract, Phellon dendron amurense Rupr extract, placenta extract, primrose extract, raspberry extract, rose extract, Rehmannia glutinosa extract, sage extract, seaweed extract, silk extract, soapwort extract, Sophora angustifolia extract, tea extract, thyme extract, thymus extract, white dead nettle extract, or any other medicinal extract.

The drug and medicinal ingredients that may be included in the composition are not limited by the above-mentioned ingredients. Drug and medicinal ingredients may be formulated alone into the composition or two or more types of medicinal ingredients may be combined and formulated suitably depending upon the objective. Further, drug and medicinal ingredients may not only be used in a free form, but may also be formulated into the composition in the form of a salt of an acid or base when capable of forming a salt or in the form of an ester when having a carboxylic acid group.

The composition may include one or more sunscreens. Examples of suitable sunscreens that may be utilized include allantoin, PABA, p-aminobenzoates, benzophenone-2, benzophenone-6, benzoresorcinol, benzyl salicylate, cinoxate, dioxybenzone, esculoside, ethyl 4-bis(hydroxypropyl)aminobenzoate, ethylhexyl p-methoxycinnamate, etocrylen, glyceryl aminobenzoate, homosalate, methyl salicylate, methyl anthranilate, methyl eugenol, 3-(4-methylbenzylidene)boran-2-one, mexenoe, octabenxone, octocrylene, oxybenzone, padimate, 2-phenyl-1H-benzimidazole-5-sulphonic acid, sulisobenzone, 3-benzylidene camphor, coffee extract, ethyl salicylate, glyceryl PABA, homosalate, isopropylbenzylsalicylate, menthyl anthranilate, nylon-12 (and) titanium dioxide, octyl dimethyl PABA, octyl methoxycinnamate, octyl salicylate, octyl triazone, orizanol, PEG-25 PABA, TEA-salicylate, titanium dioxide, zinc oxide, benzophenone-1, benzophenone-3, benzophenone-4, bensophenone-8, benzophenone-9, benzophenone-11, benzophenone-12, butyl methoxydibenzoylmethane, 4-isopropyl dibenzoyl methane, avocadin, argana oil, DEA-methoxycinnamate, drometrizole, ethyl dihydroxypropyl p-aminobenzoic acid, etocrylene, isopropyl methoxycinnamate, 3-(4-methylbenzylidene)-camphor, octocrylene, octrizole, octyl dimethyl PABA, octyl methoxycinnamate, octyl salicylate, octyl triazone, PABA, shea butter, TEA-salicylate, tri-PABA-panthenol, or any other sunscreen.

The composition may include one or more insect repellants. Examples of suitable insect repellents that may be utilized include butopyronoxyl, butylethylpropanediol, dibutyl phthalate, diethyltoluamide, dimethyl phthalate, ethohexadiol, citronella, camphor, or any other insect repellant.

The composition may include one or more preservatives. Examples of suitable preservatives that may be utilized include grape seed extract, cocoa butter, methylparaben, propylparaben, diazolindinyl urea, sorbic acid, phenoxyethanol, ethylparaben, butylparaben, sodium butylparaben, caprylyl glycol, dehydroacetic acid, or any other preservative. The composition may or may not include a preservative, and may include a plurality of preservatives. Preservatives may help to prevent undesired bacteria and fungus from developing in the composition. Preservatives may also increase the shelf life of the composition. Shelf life may refer to the time between when the composition is produced and the time the composition is applied to a skin surface. Preservatives may serve different purposes which are known to those having skill in the art.

The composition may further include sugar (e.g. white sugar, brown sugar, etc.), or a sugar equivalent, or other exfoliants or granular materials which assist in exfoliation. Examples of suitable exfoliants that may be utilized include pumus, apricot meal, ground oats, walnut shell flour, and ground almond meal. One embodiment of the composition includes white sugar.

The composition may include one or more fragrances and/or colors (e.g. pigments, dyes, etc.). Any type of natural or synthetic fragrance, such as floral, herbal or fruity fragrance could be utilized. The use of fragrance is well known in the cosmetic art and in the art of over-the-counter drug formulation, and many suitable fragrances are known in the art. The stability and function of the composition is generally not altered by the presence or absence of fragrance. Freesia essential oil may be used as a natural fragrance. Other essential oils may also be used as a natural fragrance. Fragrance can be omitted, and it may be desirable to omit fragrance in circumstances in which the composition is intended for use on sensitive individuals or individuals who may undergo an allergic reaction to fragrance. Any type of natural or FD&C colorant, such as FD&C Blue No. 1, may be utilized. Optionally, the composition may be colorless, or possess a color provided by one or more of the compounds present therein.

The composition may further include various pharmaceutically or cosmetically acceptable excipients or additives such as those which usually are employed in cosmetic or pharmaceutical compositions. Excipients or additives may be pH adjusting agents, stabilizing agents, coloring agents, foaming agents, viscosity adjusting agents, skin lightening agents such as arbutin, fillers or thickening agents such as alginate and Carbomer-940, spreading agents, pearl gloss agents, agents which protect the skin against aggressive substances in water, atmospheric air and on solid surfaces such as salts, pigments, fats, and esters, protecting agents such as chitosan, salts, waxes, and long chain alcohols. Other additives include lactic acid, citric acid, glycolic acid, succinic acid, tartaric acid, dl-malic acid, potassium carbonate, sodium hydrogencarbonate, ammonium hydrogencarbonate, and other pH adjusters. It is to be appreciated that various combinations of the aforementioned additives may be utilized in the composition of this disclosure. Moreover, the composition may be substantially free, or completely free, of such components.

The composition may be formulated to include a cosmetically acceptable carrier (or vehicle). Examples of cosmetically acceptable carriers include, but are limited to, water, glycerin, waxes, various alcohols such as ethanol, propyl alcohol, vegetable oil, mineral oil, silicones such as silicone oils, fatty esters, fatty alcohols, glycols, polyglycols or any combinations thereof. Further examples are described in the optional additives above.

The compositions of this disclosure can be prepared using various methods understood in the art. In one example of preparing the composition, the preparation method comprises the step of combining at least one of the population of microorganisms and the component obtained from the population of microorganisms, optionally along with one or more additional components (e.g. carriers and/or additives) as described above, to obtain the composition. The components can be combined using conventional manufacturing methods and apparatuses, e.g. a mixer, a blender, etc.

Finished compositions may be in any form suitable for topical application to the skin such as, but not limited to, aerosol spray, gel, cream, dispersion, emulsion, foam, liquid, lotion, moisturizer, mousse, patch, pomade, powder, pump spray, solid, solution, stick, suave, or towelette. Emulsions may include oil-in-water emulsions, water-in-oil emulsions, and water-in-silicone emulsions. In various embodiments, the composition may be used in the form of a pharmaceutical, quasi-pharmaceutical, or cosmetic. It may take the form of a lotion, cream, ointment, powder, gel, aerosol, foam, facial cleanser, balm, gel, shampoo, conditioner, wash, rinse, towelette, beauty liquid, pack, mask, makeup, foundation, scrub, exfoliant, soap, lipstick, hair cosmetic, body cosmetic, or any other suitable form for application to external surfaces of the body. The form capable of being taken by the composition is not limited to these forms however. In certain embodiments, the composition is in the form of a topical composition, optionally in the form of a topical lotion, topical wash, topical crème, topical bar, topical stick, or combinations thereof.

### INDUSTRIAL APPLICABILITY

This invention of this disclosure is useful for modulating the composition of the skin microbiome, specifically Corynebacterium kroppenstedtii spp. with postbiotics and/or probiotics. The abundance of Corynebacterium kroppenstedtii spp. on skin can also be used as a diagnostic marker for the likelihood of acquiring or having skin inflammation (either clinically visible or sub-clinical).

The following examples, illustrating the methods and compositions of this disclosure, are intended to illustrate and not to limit the invention.

### EXAMPLES

Two cross-sectional observational studies were conducted. In the first study, 495 subjects were observed. The study was a walk-in voluntary cross-sectional study conducted in mid-Michigan, early Fall. The subjects ranged from 10 to over 70 years of age. Microbiome swab sampling was at five body sites: forehead, scalp, forearm, nose, and mouth. 300+ variables were collected for each subject. In the second study, 155 subjects were observed in mid-Michigan, late Spring.

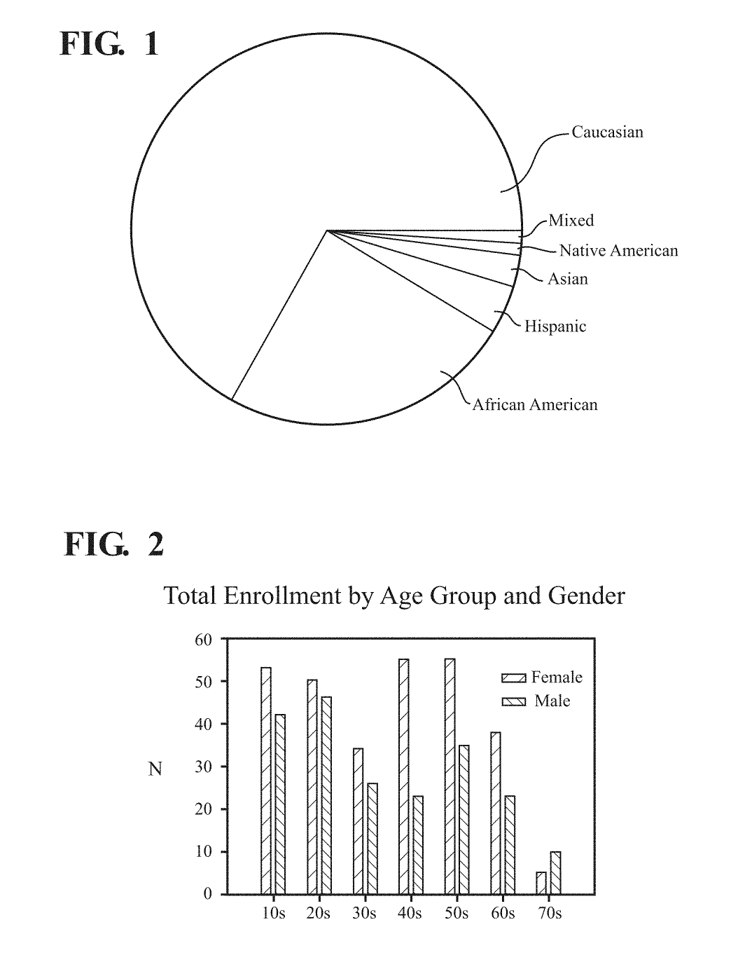

Referring to the Figures, FIG. 1 is a pie chart illustrating subject demographics of the first study. FIG. 2 is a bar chart further illustrating subject demographics of the first study.

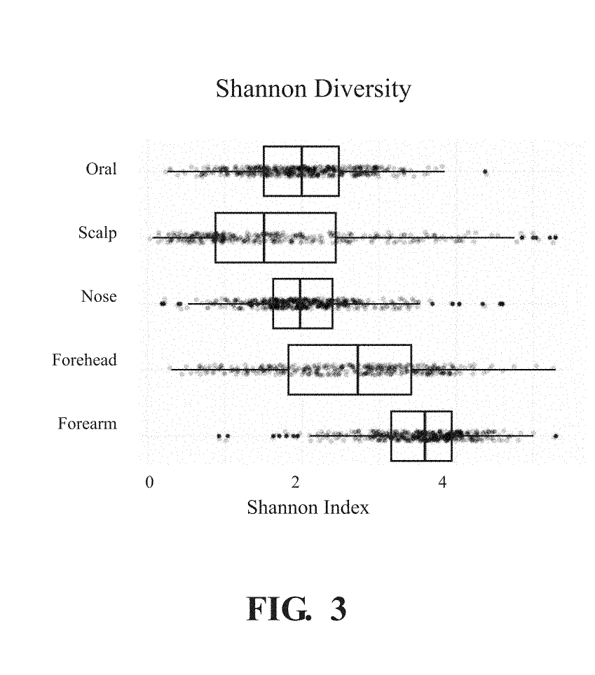

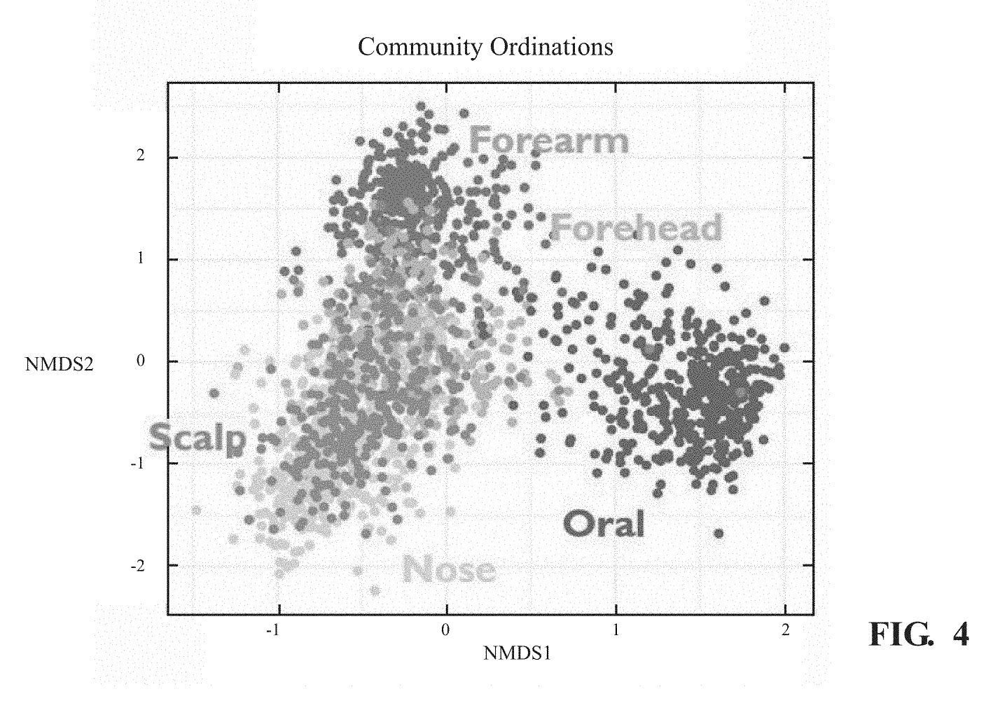

FIG. 3 is a panel illustrating bacterial diversity at each site as estimated with the Shannon Index. Each point on the graph represents the diversity score of a sample. FIG. 4 is an ordination displaying bacterial microbial composition similarity among samples. Points represent individual microbiomes, color-coded according to site.

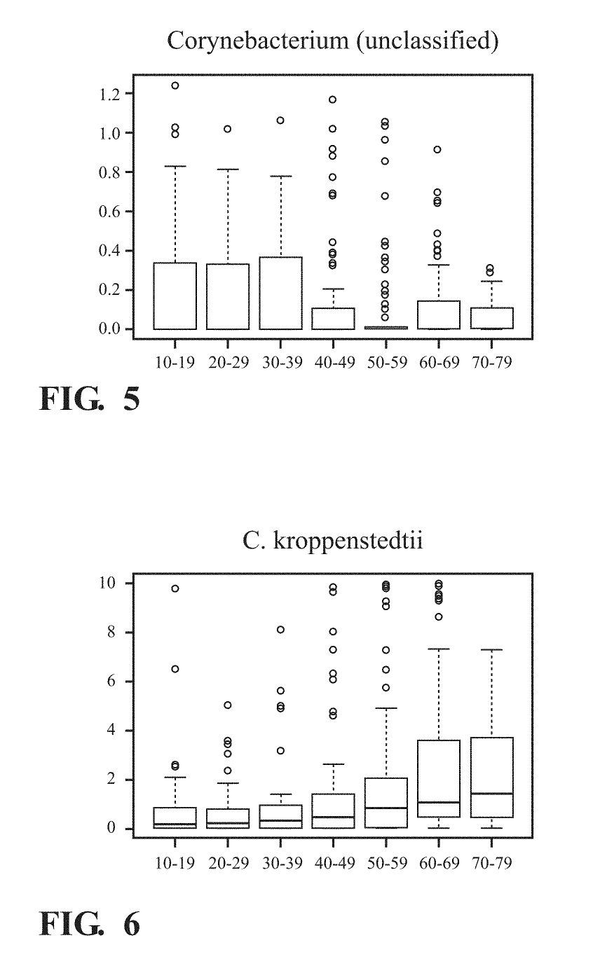

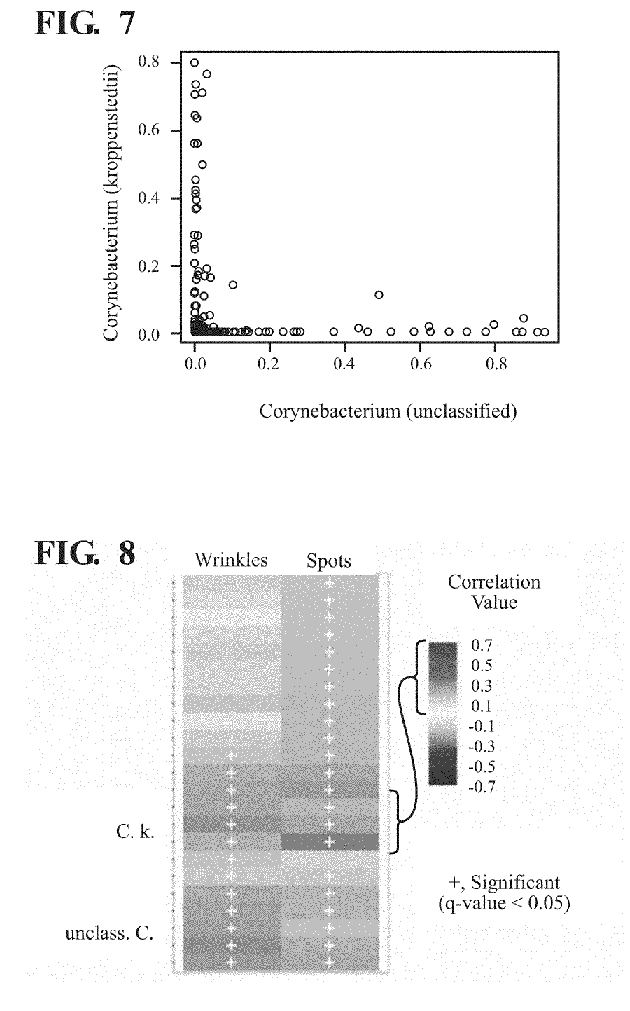

FIG. 5 is a box-and-whisker plot showing forehead species-level analysis of Corynebacterium (unclassified). FIG. 6 is a box-and-whisker plot showing forehead species-level analysis of Corynebacterium kroppenstedtii. Oligotyping pipeline was used to identify species-level variation within the genus Corynebacterium. Species-level variants were associated with Corynebacterium (unclassified) and Corynebacterium kroppenstedtii by visualizing how their relative abundances (y-axis) change with age (x-axis). Corynebacterium kroppenstedtii tended to displace Corynebacterium (unclassified) in middle-aged adults (40-49 years), in accordance with their high degree of co-exclusion (FIG. 7). FIG. 7 is a scatter chart illustrating how Corynebacterium (unclassified) and Corynebacterium kroppenstedtii are mutually exclusive.

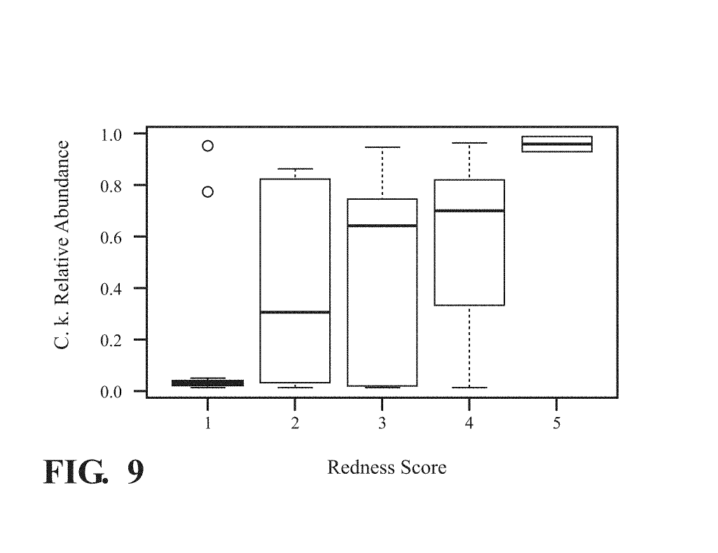

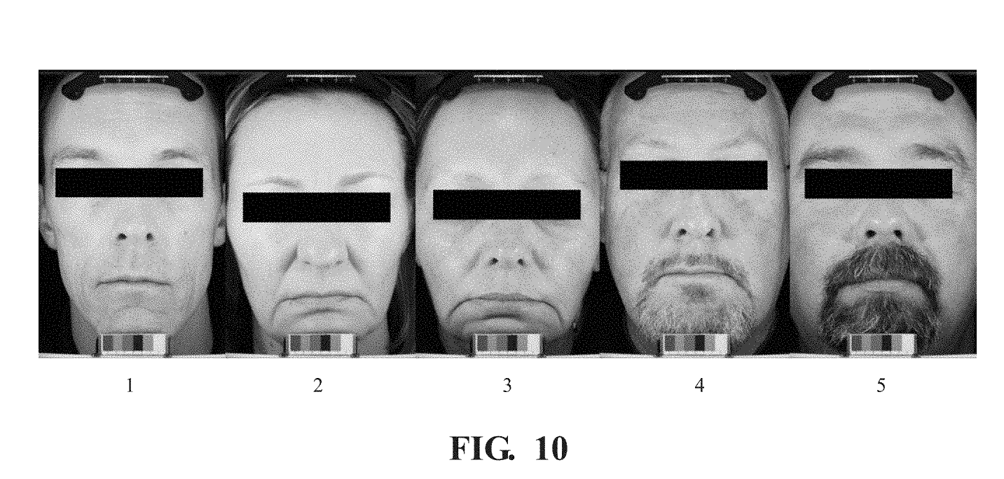

FIG. 8 is a heat map illustrating how Corynebacterium kroppenstedtii correlates with wrinkles and age spots. As also illustrated, Corynebacterium (unclassified) generally has low to no correlation with wrinkles and age spots. FIG. 9 is a box-and-whisker plot illustrating redness score distribution as a function of Corynebacterium kroppenstedtii relative abundance. FIG. 10 is a series of photos illustrating a skin redness visual grading scale, ranging from low/no redness on the left (designated as 1) to higher redness on the right (designated as 5).

Surprisingly, it was discovered that Corynebacterium (unclassified) is associated with young people and Corynebacterium kroppenstedtii is associated with older people. In addition, it was discovered that Corynebacterium (unclassified) and Corynebacterium kroppenstedtii are mutually exclusive. When one is present, the other is not (i.e., they don't coexist). Surprisingly, it was discovered that Corynebacterium kroppenstedtii is significantly associated with skin redness (r2>0.4, see FIG. 9). Since Corynebacterium (unclassified) and Corynebacterium kroppenstedtii are mutually exclusive, Corynebacterium (unclassified) can be used as a tool to modulate Corynebacterium kroppenstedtii (e.g., via culture supernatants and/or with the organism itself).

The following additional embodiments are provided, the numbering of which is not to be construed as designating levels of importance.

### ADDITIONAL EMBODIMENTS

Embodiment 1 relates to a method of modifying a skin microbiome, the method comprising: administering a topical composition to a subject's skin; wherein the topical composition comprises a population of microorganisms, a component obtained from the population of microorganisms, or a combination thereof; wherein the population of microorganisms is a Corynebacterium species; and wherein the Corynebacterium species comprises at least about 90%, optionally at least about 97%, sequence identity to a 16S rRNA sequence (SEQ ID NO: 1).

Embodiment 2 relates to Embodiment 1, wherein the topical composition comprises the population of microorganisms.

Embodiment 3 relates to Embodiment 1 or 2, wherein the topical composition comprises the component obtained from the population of microorganisms.

Embodiment 4 relates to Embodiment 3, wherein the component comprises a supernatant and/or a derivative thereof obtained from the population of microorganisms.

Embodiment 5 relates to any one of the preceding Embodiments, wherein at least one of the population of microorganisms and the component obtained from the population of microorganisms is present in the topical composition in a therapeutically effective amount to reduce, slow, and/or prevent at least one skin condition of the subject.

Embodiment 6 relates to Embodiment 5, wherein the skin condition of the subject includes inflammation, redness, hyperpigmentation, wrinkling, or combinations thereof.

Embodiment 7 relates to any one of the preceding Embodiments, wherein the topical composition is in the form of a topical pharmaceutical composition or a topical cosmetic composition.

Embodiment 8 relates to any one of the preceding Embodiments, wherein the topical composition further comprises at least one cosmetically acceptable carrier, excipient, additive, or combinations thereof.

Embodiment 9 relates to any one of the preceding Embodiments, wherein the subject is a human and is at least 18 years of age, optionally is from about 30 to about 80 years of age.

Embodiment 10 relates to any one of the preceding Embodiments, wherein the subject's skin includes a native population of microorganisms prior to administering the topical composition, and wherein the native population of microorganisms is different from the population of microorganisms associated with the topical composition administered to the subject's skin.

Embodiment 11 relates to Embodiment 10, wherein the native population of microorganisms comprises Corynebacterium kroppenstedtii, optionally wherein the Corynebacterium kroppenstedtii comprises at least about 90%, optionally at least about 97%, sequence identity to a reference 16S rRNA sequence (SEQ ID NO: 2).

Embodiment 12 relates to any one of the preceding Embodiments, wherein the topical composition is applied by hand to the subject's skin, optionally wherein the topical composition is rubbed and/or massaged on the subject's skin.

Embodiment 13 relates to any one of the preceding Embodiments, wherein the subject's skin is further defined as the subject's face, optionally is further defined as at least the subject's forehead.

Embodiment 14 relates to a topical composition for modifying a subject's skin microbiome, the topical composition comprising: a population of microorganisms, a component obtained from the population of microorganisms, or a combination thereof; wherein the population of microorganisms is a Corynebacterium species; and wherein the Corynebacterium species comprises at least about 90%, optionally at least about 97%, sequence identity to a 16S rRNA sequence (SEQ ID NO: 1).

Embodiment 15 relates to Embodiment 14, wherein the topical composition comprises the population of microorganisms.

Embodiment 16 relates to Embodiment 14 or 15, wherein the topical composition comprises the component obtained from the population of microorganisms.

Embodiment 17 relates to Embodiment 16, wherein the component comprises a supernatant and/or a derivative thereof obtained from the population of microorganisms.

Embodiment 18 relates to any one of Embodiments 14 to 17, wherein at least one of the population of microorganisms and the component obtained from the population of microorganisms is present in the topical composition in a therapeutically effective amount to reduce, slow, and/or prevent at least one skin condition of the subject.

Embodiment 19 relates to any one of Embodiments 14 to 18, wherein the topical composition is in the form of a topical pharmaceutical composition or a topical cosmetic composition.

Embodiment 20 relates to any one of Embodiments 14 to 19, wherein the topical composition further comprises at least one cosmetically acceptable carrier, excipient, additive, or combinations thereof.

Embodiment 21 relates to use of the topical composition according to any one of Embodiments 14 to 20 for a subject's skin.

Embodiment 22 relates to use of the topical composition according to any one of Embodiments 14 to 20 for addressing a skin condition selected from the group consisting of inflammation, redness, hyperpigmentation, wrinkling, and combinations thereof.

The terms “comprising” or “comprise” are used herein in their broadest sense to mean and encompass the notions of “including,” “include,” “consist(ing) essentially of,” and “consist(ing) of.” The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of ±0-10, ±0-5, or ±0-2.5, % of the numerical values. Further, The term “about” applies to both numerical values when associated with a range of values. Moreover, the term “about” may apply to numerical values even when not explicitly stated.

Generally, as used herein a hyphen “-” or dash “—” in a range of values is “to” or “through”; a “>” is “above” or “greater-than”; a “≥” is “at least” or “greater-than or equal to”; a “<” is “below” or “less-than”; and a “≤” is “at most” or “less-than or equal to.” On an individual basis, each of the aforementioned applications for patent, patents, and/or patent application publications, is expressly incorporated herein by reference in its entirety in one or more non-limiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology that has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

What is claimed is:

1. A method of modifying a skin microbiome, the method comprising:
administering a topical composition to a subject's skin;
wherein the topical composition comprises a population of microorganisms, a component obtained from the population of microorganisms, or a combination thereof;
wherein the population of microorganisms is a Corynebacterium species; and
wherein the Corynebacterium species comprises at least about 90%, optionally at least about 97%, sequence identity to a 16S rRNA sequence (SEQ ID NO: 1).

2. The method according to claim 1, wherein the topical composition comprises the population of microorganisms.

3. The method according to claim 1, wherein the topical composition comprises the component obtained from the population of microorganisms.

4. The method according to claim 3, wherein the component comprises a supernatant and/or a derivative thereof obtained from the population of microorganisms.

5. The method according to claim 1, wherein at least one of the population of microorganisms and the component obtained from the population of microorganisms is present in the topical composition in a therapeutically effective amount to reduce, slow, and/or prevent at least one skin condition of the subject.

6. The method according to claim to 5, wherein the skin condition of the subject includes inflammation, redness, hyperpigmentation, wrinkling, or combinations thereof.

7. The method according to claim 1, wherein the topical composition is in the form of a topical pharmaceutical composition or a topical cosmetic composition.

8. The method according to claim 1, wherein the topical composition further comprises at least one cosmetically acceptable carrier, excipient, additive, or combinations thereof.

9. The method according to claim 1, wherein the subject is a human and is at least 18 years of age, optionally is from about 30 to about 80 years of age.

10. The method according to claim 1, wherein the subject's skin includes a native population of microorganisms prior to administering the topical composition, and wherein the native population of microorganisms is different from the population of microorganisms associated with the topical composition administered to the subject's skin.

11. The method according to claim 10, wherein the native population of microorganisms comprises Corynebacterium kroppenstedtii, optionally wherein the Corynebacterium kroppenstedtii comprises at least about 90%, optionally at least about 97%, sequence identity to a reference 16S rRNA sequence (SEQ ID NO: 2).

12. The method according to claim 1, wherein the topical composition is applied by hand to the subject's skin, optionally wherein the topical composition is rubbed and/or massaged on the subject's skin.

13. The method according to claim 1, wherein the subject's skin is further defined as the subject's face, optionally is further defined as at least the subject's forehead.

14. A topical composition for modifying a subject's skin microbiome, the topical composition comprising:
a population of microorganisms, a component obtained from the population of microorganisms, or a combination thereof;
wherein the population of microorganisms is a Corynebacterium species; and
wherein the Corynebacterium species comprises at least about 90%, optionally at least about 97%, sequence identity to a 16S rRNA sequence (SEQ ID NO: 1).

15. The topical composition according to claim 14, wherein the topical composition comprises the population of microorganisms.

16. The topical composition according to claim 14, wherein the topical composition comprises the component obtained from the population of microorganisms.

17. The topical composition according to claim 16, wherein the component comprises a supernatant and/or a derivative thereof obtained from the population of microorganisms.

18. The topical composition according to claim 14, wherein at least one of the population of microorganisms and the component obtained from the population of microorganisms is present in the topical composition in a therapeutically effective amount to reduce, slow, and/or prevent at least one skin condition of the subject.

19. The topical composition according to claim 14, wherein the topical composition is in the form of a topical pharmaceutical composition or a topical cosmetic composition.

20. The topical composition according to claim 14, wherein the topical composition further comprises at least one cosmetically acceptable carrier, excipient, additive, or combinations thereof.


SEQUENCE LISTING (2 sequences)

SEQ ID NO: 1 - DNA, 254 bp, Corynebacterium (unclassified)
tacgtagggtgcgagcgttgtccggaattactgggcgtaaagggctcgtaggtggtttgtcgcgtcgtctgtgaaattccggggcttaactccgggcgtgcaggcgatacgggcataacttgagtactgtaggggtaactggaattcctggtgtagcggtgaaatgcgcagatatcaggaggaacaccgatggcgaaggcaggttactgggcagttactgacgctgaggagcgaaagcatgggtagcgaacagg

SEQ ID NO: 2 - DNA, 254 bp, Corynebacterium kroppenstedtii
tacgtagggtgcgagcgttgtccggaattactgggcgtaaagagctcgtaggtggtctgtcgcgtcatttgtgaaagcccggggcttaactccgggttggcaggtgatacgggcatgactggagtactgtaggggagactggaattcctggtgtagcggtgaaatgcgcagatatcaggaggaacaccggtggcgaaggcgggtctctgggcagtaactgacgctgaggagcgaaagcatgggtagcgaacagg